(19) United States
(12) Patent Application Publication
(10) Pub. No.: US 2021/0085896 A1
(43) Pub. Date: Mar. 25, 2021

(54) MOUTHPIECE FOR USE WITH NEBULIZER AND SIMILAR DEVICES

(71) Applicant: Spencer Boatman, Granada Hills, CA (US)

(72) Inventor: Spencer Boatman, Granada Hills, CA (US)

(21) Appl. No.: 17/030,261

(22) Filed: Sep. 23, 2020

Related U.S. Application Data
(60) Provisional application No. 62/878,324, filed on Jul. 24, 2019.

(51) Int. Cl. A61M 15/00

(52) U.S. Cl. CPC A61M 15/0021; A61M 15/009

(57) ABSTRACT

Described herein are improved mouthpieces for the delivery of air (sometimes including medicine) to a user. The mouthpieces can include features that enable the user to better keep the mouthpiece in his or her mouth, such as one or more ridges and/or a wider mouth portion; can include features for increased durability, such as one or more internal support pillars; and can include combinations of these features.

The present application claims priority to U.S. Provisional Pat. App. No. 62/878,324 to Boatman, filed on Jul. 24, 2019, entitled “MOUTHPIECE FOR USE WITH NEBULIZER AND SIMILAR DEVICES,” the entire contents of which are hereby incorporated by reference.

### BACKGROUND OF THE DISCLOSURE

### Field of the Disclosure

Embodiments of the present disclosure relate to mouthpieces that more easily remain within a user's mouth, and/or that are more durable. More specific embodiments of the present disclosure relate to improved mouthpieces for use with nebulizers, and in some instances for use with nebulizers in combination with one or more additional therapeutic and/or medicinal devices.

### Related Art

Nebulizers are used to change medication from a liquid to a mist so that the medication can be inhaled by a user. They are often used for treatment of respiratory ailments such as asthma. The nebulizer is connected to a mouthpiece, through which a user can inhale air including the medicine.

Additional devices are sometimes included between the nebulizer and the mouthpiece. For instance, positive expiratory pressure (PEP) devices can be included. These devices can have additional benefits to the user, such as clearance of excess lung secretion and improved lung endurance, but add weight to the overall system. One example of such a device is the Aerobika™ Oscillating PEP Therapy System.

FIG. 1A shows a typical nebulizer mouthpiece 10, while FIG. 1B shows the mouthpiece 10 in combination with a PEP device 50. The mouthpiece 10 can be hooked directly up to a nebulizer, or as shown in FIG. 1B can be hooked up to an additional device such as the PEP device 50, and then the opposite end 52 of the PEP device is hooked up to the nebulizer. The mouthpiece 10 includes a mouth portion 12 and a connection portion 14. The mouth portion 12 has a horizontally elongated cross section including a flat top and a flat bottom connected by semicircular ends. The connection portion 14 is generally tubular in nature, with a diameter that is larger than the height of the mouth portion but smaller than the width of the mouth portion. Typical dimensions of the mouth piece 10 are shown in FIG. 1C. Typically about one half of the depth of the mouthpiece 10 is the mouth portion 12, while the other half of the depth is the connection portion 14.

### SUMMARY OF THE DISCLOSURE

Described herein are embodiments of mouthpieces that are more easily retained in a user's mouth and/or which include one or more features for improved durability.

In one embodiment of the disclosure, a mouthpiece includes a connection portion and a mouth portion for insertion into a user's mouth connected to the connection portion, the mouth portion comprising one or more ridges configured to abut a user's teeth.

In another embodiment of the disclosure, a mouthpiece includes a connection portion defining a connection portion airway therethrough, and a mouth portion for insertion into a user's mouth connected to the connection portion, the mouth portion defining a mouth portion airway therethrough that is connected to the connection portion airway. The mouthpiece further includes at least one support pillar in said mouth portion airway.

In yet another embodiment of the disclosure, a mouthpiece includes a connection portion and a mouth portion for insertion into a user's mouth connected to the connection portion, the mouth portion comprising one or more ridges configured to abut a user's teeth. The connection portion defines a connection portion airway therethrough, and the mouth portion defines a mouth portion airway therethrough that is connected to the connection portion airway. The mouthpiece further includes at least one support pillar in the mouth portion airway.

These and other further features and advantages of the disclosure would be apparent to those skilled in the art from the following detailed description.

### BRIEF DESCRIPTION OF THE DRAWINGS

FIG. 1A is a perspective view of a prior art mouthpiece, and FIG. 1B is a perspective view of the prior art mouthpiece in combination with a PEP device.

FIG. 1C shows various views of a prior art mouthpiece and typical prior art dimensions of such a mouthpiece.

FIG. 2A is a perspective view of a mouthpiece according to the present disclosure, and FIG. 2B is a side view of the mouthpiece shown in FIG. 2A.

FIG. 3 is a side view of another embodiment of a mouthpiece according to the present disclosure.

FIG. 4A is a perspective view of a mouthpiece according to yet another embodiment the present disclosure, and FIG. 4B is a side view of the mouthpiece shown in FIG. 4A.

FIG. 5 is a perspective view of a mouthpiece according to yet another embodiment of the present disclosure.

FIG. 6A is a top view of a prior art mouthpiece, and FIG. 6B is a top view of yet another embodiment of a mouthpiece according to the present disclosure.

FIGS. 7A-7D are perspective, side, top, and rear views, respectively, of a mouthpiece according to yet another embodiment of the present disclosure.

FIGS. 8A-8E are perspective, side, top, rear, and front views, respectively, of a mouthpiece according to yet another embodiment of the present disclosure.

### DETAILED DESCRIPTION OF THE DISCLOSURE

Embodiments of the present disclosure are directed to improved mouthpieces for the delivery of air, and particularly air including medicine, to a user. Embodiments can include features that enable a user to better keep the mouthpiece in his or her mouth, such as one or more ridges, and/or a wider mouth portion; and/or features that can lead to increased durability, such as one or more internal support pillars.

Embodiments of the disclosure are described herein with reference to illustrations that are schematic illustrations of embodiments of the disclosure. As such, the actual size, components and features can be different, and variations from the shapes of the illustrations as a result, for example, of technological capabilities, manufacturing techniques and/or tolerances are expected. Embodiments of the disclosure should not be construed as limited to the particular shapes or components of the regions illustrated herein but are to include deviations in shapes/components that result, for example, from manufacturing or technological availability. The regions illustrated in the figures are schematic in nature and their shapes are not intended to illustrate the precise shape or functionality of a feature of a device and are not intended to limit the scope of the disclosure. In addition, components may be shown as one unit but may instead be a collection of components or units, or a collection of components or units may exist as one unit.

Throughout this description, the preferred embodiment and examples illustrated should be considered as exemplars, rather than as limitations on the present disclosure. As used herein, the term “disclosure,” “device,” “method,” “present disclosure,” “present device” or “present method” refers to any one of the embodiments of the disclosure described herein, and any equivalents. Furthermore, reference to various feature(s) of the “disclosure,” “device,” “method,” “present disclosure,” “present device” or “present method” throughout this document does not mean that all claimed embodiments or methods must include the referenced feature(s).

It is also understood that when an element or feature is referred to as being “on” or “adjacent” another element or feature, it can be directly on or adjacent to the other element or feature, or intervening elements or features may also be present. It is also understood that when an element is referred to as being “connected” or “coupled” to another element, it can be directly connected or coupled to the other element, or intervening elements may be present. In contrast, when an element is referred to as being “directly connected” or “directly coupled” to another element, there are no intervening elements present.

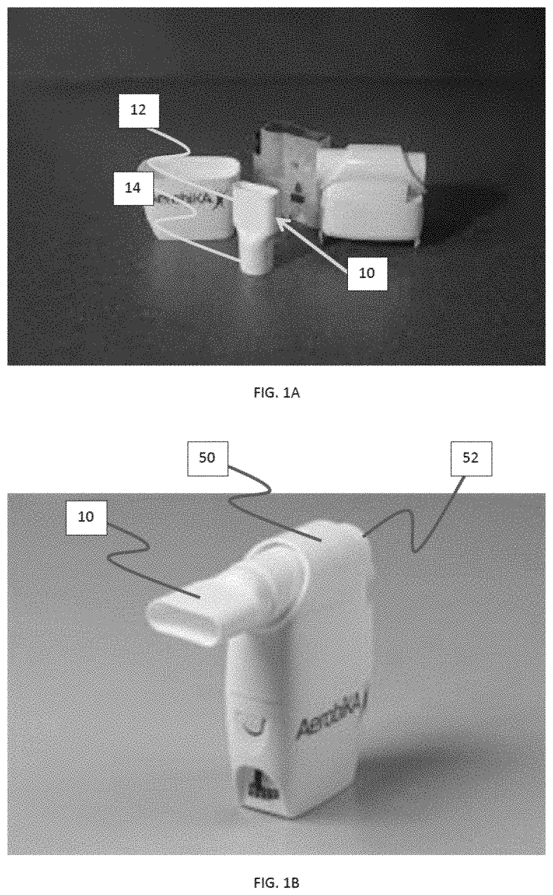

Relative terms such as “outer,” “above,” “lower,” “below,” “horizontal,” “vertical” and similar terms, may be used herein to describe a relationship of one feature to another. It is understood that these terms are intended to encompass different orientations in addition to the orientation depicted in the figures. In relation to the mouthpieces of the present disclosure, the general direction to which “width” as used herein refers to is the direction of the 32 mm dimension in FIG. 1C; the general direction to which “height” as used herein refers to is the direction of the 13 mm dimension in FIG. 1C, and the general direction to which “depth” as used herein refers to is the direction of the 57 mm dimension in FIG. 1C.

Although the terms first, second, etc. may be used herein to describe various elements or components, these elements or components should not be limited by these terms. These terms are only used to distinguish one element or component from another element or component. Thus, a first element or component discussed below could be termed a second element or component without departing from the teachings of the present disclosure. As used herein, the term “and/or” includes any and all combinations of one or more of the associated list items.

The terminology used herein is for describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” when used herein, specify the presence of stated features, integers, steps, operations, elements, and/or components, but do not preclude the presence or addition of one or more other features, integers, steps, operations, elements, components, and/or groups thereof.

Current mouthpieces used for the delivery of air, and especially air including medicine such as mouthpieces used with nebulizers, suffer from various flaws. First, users may attempt to use such mouthpieces without support from their hands and/or only with support from their mouth, leaving their hands free for other activities such as reading a book. However, prior art mouthpieces are not shaped to accommodate such a scenario, and the mouthpiece can often slip or fall out of a user's mouth, or move to an uncomfortable or improper position, due to the weight of devices on the other end of the mouthpiece (such as a nebulizer and/or PEP device). Second, because the portion of the mouthpiece that is designed to go into a user's mouth (the “mouth portion”) is often held in place by substantial force from the user's mouth, the airway through the mouth portion can partially or fully collapse, and/or the mouth portion can become deformed. This can make the passage of air and/or holding the mouth portion in a user's mouth more difficult.

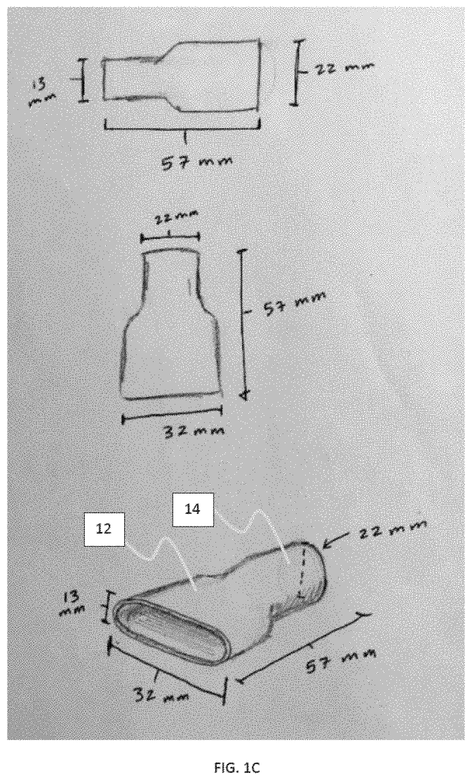

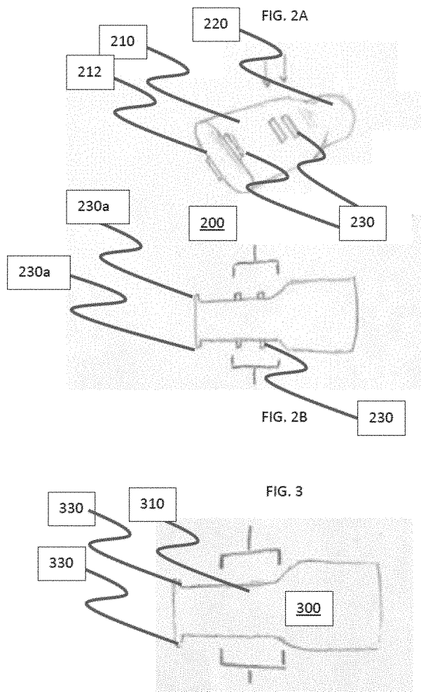

FIGS. 2A and 2B show one embodiment of a mouthpiece according to the present disclosure. The mouthpiece 200 includes a mouth portion 210 and a connection portion 220, each with an airway therethrough. The mouth portion 210 comprises three ridges 230 on its top surface and three ridges 230 on its bottom surface, with one ridge 230a on each surface on the front end 212 of the mouth portion 210 and/or top or bottom surface. The ridges can be configured to abut the rear of a user's teeth such that the mouthpiece 200 is held in the user's mouth. It is understood that any single ridge 230 or combination of the shown ridges 230 is possible. Embodiments according to the present disclosure can include one ridge, two ridges, three ridges, four ridges, five ridges, six ridges, or more. One example of a mouthpiece 300 including only two ridges 330, one on the front end of each of the top and bottom surfaces of the mouth portion 310, is shown in FIG. 3.

Some embodiments do not include ridges on the front end 212 of the mouth portion 212. The ridges on that are further rearward on the mouth portion 210 can be useful in that they can provide better leverage for a user that is using his or her teeth/mouth to support devices on the other end of the mouthpiece 200. Including two or more such ridges, such as two or more such ridges on the top surface of the mouth portion 210 and two or more ridges on the bottom surface, can be useful in that users with different mouth sizes (e.g., children versus adults) can use different ridges. Additionally, in some embodiments the ridges can be placed in proximity to one another such that a user's teeth will fit between the ridges, such as within 1/16″ or ⅛″ or one another.

Ridges according to the present disclosure can run substantially in the width direction of the mouth portion 210, and can run along substantially the entire width of the top and/or bottom surface, or along less than all the width of the top and/or bottom surface.

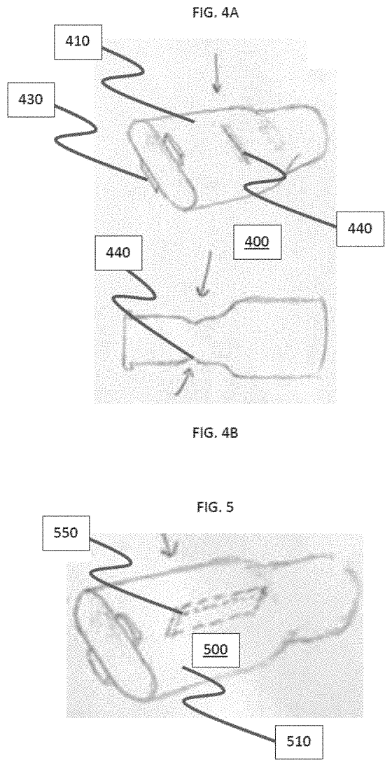

While “ridges” as used herein refers to positive space elements such as protrusions, it is understood that other elements are possible, such as indentations. FIGS. 4A and 4B show an embodiment of a mouthpiece 400 including a mouth portion 410 with front end ridges 430 and rearward indentations 440. It is understood that in embodiments described throughout this specification, indentations could be substituted for ridges and provide some of the same benefits, or different benefits. Any combination of ridges and indentations is possible.

Additionally, while FIGS. 4A and 4B show relatively discrete indentations 440, in another embodiment the mouth portion tapers to a smaller height over a distance and then tapers back to the full height over a distance. The full distance spread out indentation, or valley, could be approximately one eighth the depth of the mouth portion or more, one fourth the depth of the mouth portion or more, one third the depth of the mouth portion or more, and/or one half the depth of the mouth portion or more.

FIG. 5 shows another embodiment of a mouthpiece 500 according to the present disclosure. The mouthpiece 500 includes a support pillar 550 in the airway of the mouth portion 510. The support pillar can help to prevent structural failure of the mouth portion 510 due to the pressure applied from a user's mouth. The support pillar 550 is shown running approximately parallel to the depth of the mouthpiece 500 and approximately in the center of the width of the mouth portion 510, though other embodiments are possible. Multiple support pillars running along the depth direction are possible, and/or support pillars can run parallel to one another, and/or support pillars can be non-central with respect to the width of the device. Many different embodiments are possible.

In some embodiments, one or more of the ridges described herein can overlap one or more of the support pillars described herein. This can be beneficial in that a user's teeth will typically abut the ridge, and thus this will be the area where the most pressure is applied to the mouth portion and where failure would be most likely, but that a support pillar is under the ridge to combat this failure.

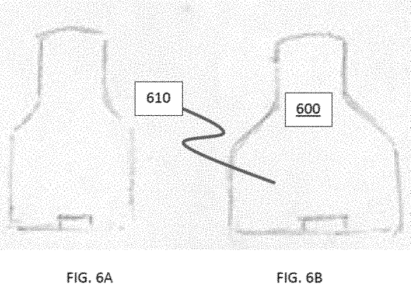

Another embodiment of a mouthpiece 600 according to the present disclosure is shown in FIG. 6B. The prior art mouthpiece shown in FIG. 6A has approximately the dimensions of the prior art mouthpiece shown in FIG. 1C. However, the mouthpiece 600 can include a wider mouth portion 610. This can aid in supporting the mouthpiece 600 because, for instance, more teeth may be in contact with the mouth portion, spreading out the pressure caused by gravity (such as that from other devices on the other end of the connection portion as previously described). In some embodiments, the width of the mouth portion 610 of the mouthpiece 600 is 40 mm or more, or 45 mm or more, or 50 mm or more, or 55 mm or more, or 60 mm or more, or 65 mm or more, or 70 mm or more, or 80 mm or more, or 100 mm or more, or even wider. In some embodiments, the width is between 40 mm and 80 mm, or between 45 mm and 70 mm, or between 50 mm and 60 mm. In the prior art, the width to height ratio is approximately 2.5. In some embodiments of the present disclosure, this ratio is 3 or higher, or 3.25 or higher, or 3.5 or higher, or 3.75 or higher, or 4 or higher, or 4.25 or higher, or 4.5 or higher, or 5 or higher, or 5.5 or higher, or 6 or higher, or 6.5 or higher, or 7 or higher, or 7.5 or higher, or even higher.

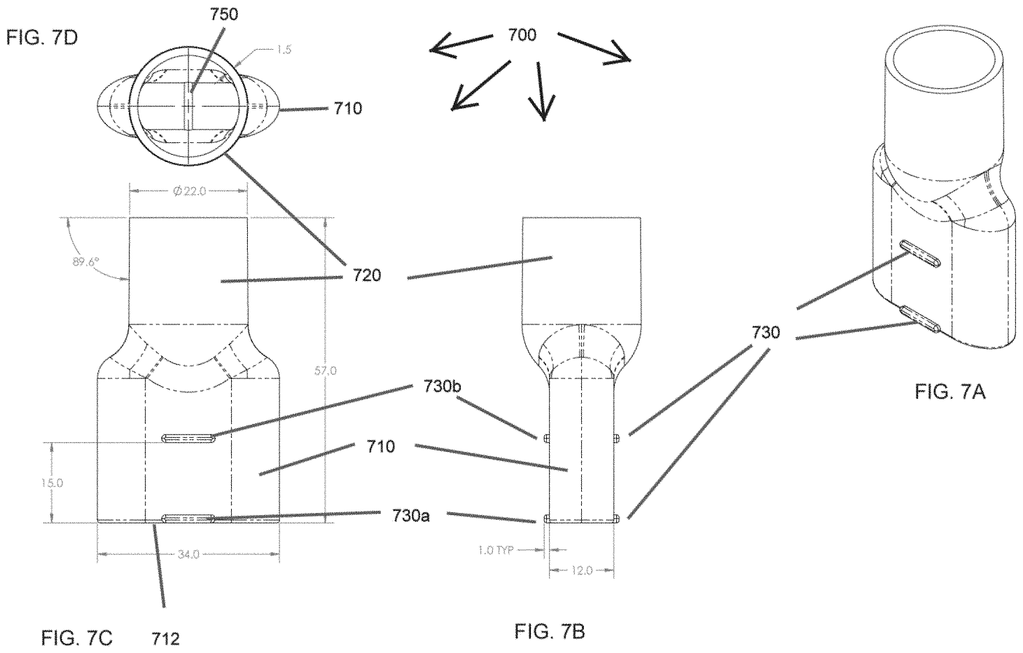

Another embodiment of a mouthpiece 700 according to the present disclosure is shown in FIGS. 7A-7D. The mouthpiece 700 includes a mouth portion 710 with front end 712, a connection portion 720, and ridges 730. In this specific embodiment, four ridges 730 are included—two on the top surface of the mouth portion 710, and two on the bottom surface of the mouth portion 710, with the ridges 730 on the top surface vertically aligned with the ridges 730 on the bottom surface. It is understood that in some embodiments, the ridges 730 may not be aligned with each other, such as the top ridge 730b being more rearward than the bottom ridge 730b, which can sometimes be more comfortable to a user due to the fact that a user's top teeth are typically forward of the user's bottom teeth.

In the specific embodiment shown, the ridges 730 comprise two ridges 730a that are on the front end 712 of the mouth portion 710, and two ridges 730b that are rearward of the front end 712. In the specific embodiment shown, the ridges 730b can be 2.5 mm to 30 mm rearward of the front end 712, or 5 mm to 25 mm rearward of the front end 712, or 8 mm to 20 mm rearward of the front end 712, or 5 mm or more from the front end 712, or 8 mm or more from the front end 712, or 10 mm or more from the front end, or 25 mm or less from the front end 712, or 20 mm or less from the front end 712, or approximately 15 mm rearward of the front end 712. Many different embodiments, including those outside of these ranges, are possible. The ridges can be 0.25 mm to 3 mm high, or 0.5 mm to 2 mm high, or 0.5 mm or more high, or 3 mm or less high, or approximately 1 mm high. Many different embodiments, including those outside of these ranges, are possible. Additionally, it is understood that any combination of the ridges 730 is possible, such as only ridges 730 on the top surface, only ridges 730 on the bottom surface, only front ridges 730a, only rear ridges 730b, any three of the four ridges 730, etc.; many different combinations are possible as would be understood by one of skill in the art.

The mouthpiece 700 can also include a support pillar 750, which can be similar in many respects to the support pillar 550 described above with regard to FIG. 5. The support pillar 750 can run similarly to the support pillar 550, that is, in the frontward/rearward direction, or can run in a different direction; or, alternatively, may not run in any direction, but instead may be a post-type structure. In some embodiments, a combination of post-type structures can be used. Some embodiments include multiple support pillars such as the support pillar 750. Many different embodiments are possible.

The support pillar 750 can overlap with the ridges 730b. The support pillar can be 0.25 mm to 5 mm wide, 0.5 mm to 3 mm wide, 1 mm to 2 mm wide, or less than 5 mm wide, or less than 3 mm wide, or less than 2 mm wide, or about 1.5 mm wide, though it is understood that embodiments outside of these ranges are possible. A narrower support pillar can result in the easier passage of air past the support pillar and through the mouth portion 710, while a wider support pillar can result in more support for the mouth portion 710. The support pillar 710 (or in some instances, the combination of support pillars) can have a length of, for instance, 1 mm to 25 mm, 5 mm to 20 mm, 8 mm to 18 mm, 10 mm to 16 mm, or about 12 mm to about 14 mm, or about 12 mm or about 14 mm. Embodiments outside these ranges are possible. The support pillar 710 can have its front end at the front end 712 of the mouth portion, or offset from the front end 712 by 1 mm to 25 mm, 2 mm to 20 mm, 5 mm to 17 mm, 8 mm to 14 mm, or about 11 mm, though it is understood that different embodiments are possible. Having the support pillar 710 at or near the front end 722 can result in greater support, while having the support pillar 710 further rearward can result in better air flow for the user. In some embodiments, the support pillar 710 is entirely within the mouth portion 710; in some embodiments, the support pillar is entirely within the mouth portion 710 and a transition between the mouth portion 710 and connection portion 720; in some embodiments, the support pillar can be within both the mouth portion 710 and the connection portion 720; many different embodiments are possible.

It is understood that the dimensions shown in FIGS. 7A-7D (shown in millimeters), as well as the ratios between those dimensions, are purely exemplary in nature. Dimensions other than those shown are within the scope of the disclosure, such as the shown dimensions ±5%, ±10%, ±15%, ±25%, ±50%, or other dimensions/ratios.

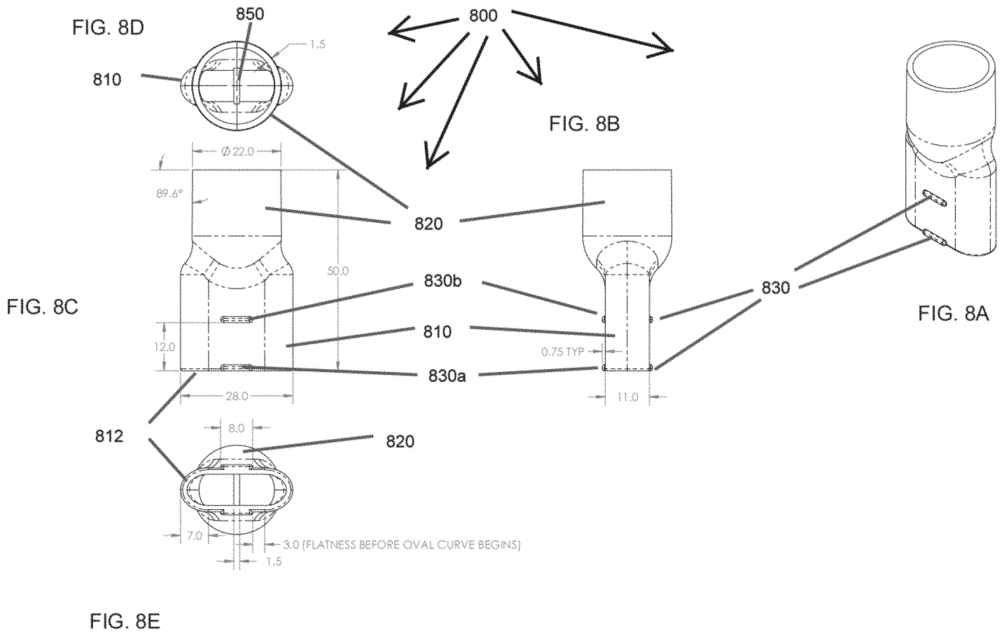

Another embodiment of a mouthpiece 800 according to the present disclosure is shown in FIGS. 8A-8E. The mouthpiece 800 includes a mouth portion 810 with front end 812 (similar to the mouth portion 710 and front end 712), a connection portion 820 (similar to the connection portion 720), ridges 830 (similar to ridges 730), and internal support 850 such as a pillar (similar to the internal support 750). The mouthpiece 800 includes smaller exemplary dimensions than the mouthpiece 700 from FIGS. 7A-7D, and thus can be better fitted for those with smaller mouths, such as, for example, children. It is understood that the dimensions shown in FIGS. 8A-8E (shown in millimeters), as well as the ratios between those dimensions, are purely exemplary in nature. Dimensions other than those shown are within the scope of the disclosure, such as the shown dimensions ±5%, ±10%, ±15%, ±25%, ±50%, or other dimensions/ratios.

Although the present disclosure has been described in detail with reference to certain preferred configurations thereof both in the specification and in the claims, other versions are possible. Embodiments of the present disclosure can comprise any combination of compatible devices/features described herein and/or shown in the figures, and these embodiments should not be limited to those expressly illustrated and discussed. Therefore, the spirit and scope of the disclosure should not be limited to the versions described above.

I claim:

1. A mouthpiece, comprising:
a connection portion; and
a mouth portion for insertion into a user's mouth connected to said connection portion, said mouth portion comprising one or more ridges configured to abut a user's teeth.

2. The mouthpiece of claim 1, comprising at least two of said ridges.

3. The mouthpiece of claim 2, wherein said mouth portion comprises a top surface and a bottom surface; and
wherein at least one of said ridges is on said top surface and at least one of said ridges is on said bottom surface.

4. The mouthpiece of claim 3, wherein said top surface is substantially flat and said bottom surface is substantially flat.

5. The mouthpiece of claim 3, wherein at least two of said ridges are on said top surface and at least two of said ridges are on said bottom surface.

6. The mouthpiece of claim 3, wherein one of said ridges on said top surface and one of said ridges on said bottom surface are substantially vertically aligned.

7. The mouthpiece of claim 1, wherein at least one of said ridges is on a front end of said mouth portion.

8. The mouthpiece of claim 1, further including a support pillar within said mouth portion.

9. The mouthpiece of claim 8, wherein said support pillar overlaps one or more of said ridges.

10. The mouthpiece of claim 9, wherein said support pillar and at least one of said overlapping ridges run perpendicularly to one another.

11. A mouthpiece, comprising:
a connection portion defining a connection portion airway therethrough; and
a mouth portion for insertion into a user's mouth connected to said connection portion, said mouth portion defining a mouth portion airway therethrough that is connected to the connection portion airway; and
at least one support pillar in said mouth portion airway.

12. The mouthpiece of claim 11, wherein said support pillar is from a top of said mouth portion airway to a bottom of said mouth portion airway.

13. The mouthpiece of claim 11, wherein said support pillar has a depth and a width, said depth larger than said width.

14. The mouthpiece of claim 13, wherein said support pillar crosses a midpoint of said mouth portion airway.

15. The mouthpiece of claim 11, wherein a front of said mouth portion airway is free of any support pillars.

16. The mouthpiece of claim 11, wherein said at least one support pillar comprises two support pillars.

17. The mouthpiece of claim 11, wherein said support pillar is approximately in a center of a width of said mouth portion.

18. The mouthpiece of claim 11, wherein said support pillar runs substantially parallel to a depth of said mouth portion.

19. The mouthpiece of claim 11, wherein said mouth portion has a width to height ratio of 3 or higher.

20. A mouthpiece for use with a nebulizer, comprising:
a connection portion defining a connection portion airway therethrough; and
a mouth portion for insertion into a user's mouth connected to said connection portion, said mouth portion defining a mouth portion airway therethrough that is connected to the connection portion airway; and
at least one support pillar in said mouth portion airway;
wherein said mouth portion comprises a top surface and a bottom surface, a first ridge on a front end of said top surface, a second ridge rearward of said first ridge on said top surface, a third ridge on a front end of said bottom surface, and a fourth ridge rearward of said third ridge on said bottom surface.
